(19) United States
(12) Patent Application Publication
(10) Pub. No.: US 2007/0196344 A1
(43) Pub. Date: Aug. 23, 2007

(54) Methods for identifying materials that can help regulate the condition of mammalian keratinous tissue

(75) Inventors: Rosemarie Osborne, Oxford, OH (US); Lisa Ann Mullins, West Chester, OH (US); Sara Johnson McPhall, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/655,347

(22) Filed: Jan. 19, 2007

Related U.S. Application Data
(60) Provisional application No. 60/760,649, filed on Jan. 20, 2006.

(51) Int. Cl. A61K 8/18; G06Q 50/00

(52) U.S. Cl. 424/93.7; 435/1.1; 705/500

(57) ABSTRACT

Methods for identifying materials that can help regulate the condition of mammalian keratinous tissue (e.g., skin, hair, and/or nails), and the use of such materials selected by such methods (e.g., preparation of cosmetic compositions). The methods can also be used to generate data that can be used to support marketing claims and advertising claims, and to provide irritation and/or safety information. The methods can be used as an element of a method of doing business.

### CROSS-REFERENCE TO RELATED APPLICATION

This application claims the benefit of priority to U.S. Provisional Application Ser. No. 60/760,649, filed Jan. 20, 2006, which is herein incorporated by reference in its entirety.

### FIELD of the Invention

The present invention relates to methods for identifying materials that can help regulate the condition of mammalian keratinous tissue (e.g., skin, hair, and/or nails). It also relates to the use of such materials selected by such methods, for preparing at least one cosmetic composition.

The methods can also be used to generate data that can be used to support marketing claims and advertising claims, and to provide irritation and/or safety information, as well as dose response information. The methods can be used as an element of a method of doing business.

### BACKGROUND OF THE INVENTION

Many personal care products currently available to consumers are directed primarily to improving the health and/or physical appearance of the skin, hair, or nails. Among these skin, hair, or nail care products, many are directed to delaying, minimizing or even eliminating skin, hair, or nail changes typically associated with the aging or the environmental damage to human skin, hair, or nails. Numerous compounds have been described in the art as being useful for regulating skin, hair, or nail condition.

Skin, hair, and nails are subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin, hair, or nails. Whether extrinsic or intrinsic, these factors result in visible signs of skin, hair, and nail aging and environmental damage (e.g., such as sunlight damage, smoke damage, and damage from pollutants such as nitrogen oxides, sulfur oxides, ozone, and metals such as lead). To many people, the loss of the attractiveness of skin, hair, or nails is a reminder of the disappearance of youth. As a result, the maintenance of a youthful appearance has become a booming business in youth-conscious societies. Numerous products and treatments are available in various forms to help maintain the appearance of younger hair, skin, and nails.

Extrinsic or intrinsic factors may result in the thinning and general degradation of the skin, hair, or nails. For example, as the skin, hair, and nails naturally age, there is a reduction in the cells and blood vessels that supply the skin, hair, or nails. There is also a flattening of the dermal-epidermal junction which results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990.

A large number of skin, hair, and nail care actives are known in the art and used to improve the health and/or cosmetic appearance of the skin, hair, or nails. However, a need still exists for additional actives that can provide the desired benefits and can be dermopharmaceutically and/or cosmetically preferred for particular applications. Identifying such suitable actives can be a laborious process. Hundreds or even thousands of materials may be tested in vivo before a suitable active is identified that gives the desired result. Thus, identifying a new active can involve a large investment of time and expense. Accordingly, it would be desirable to provide methods that can be used to quickly and accurately screen for and/or identify active materials having the desired benefits.

### SUMMARY OF THE INVENTION

The present invention relates to methods for identifying active materials that can help regulate the condition of mammalian keratinous tissue. It also relates to the materials identified by such methods, as well as the use of such materials selected by such methods, for preparing at least one cosmetic composition. Furthermore, these methods can be used to generate data that can be used to support marketing claims and/or advertising claims, and to provide irritation and/or safety information, as well as dose response information. The methods can also be used as an element of a method of doing business.

In one embodiment, the method for identifying active materials that can regulate the condition of mammalian keratinous tissue comprises the steps of:

(a) identifying a biomarker that is indicative of mammalian keratinous tissue regulation;

(b) screening in vitro at least one unknown material, preferably a plurality of unknown materials, against said biomarker to identify one or more potentially active materials;

(c) testing said one or more potentially active materials in vivo to confirm the activity of said potentially active materials in order to identify one or more active materials;

(d) optionally, validating the accuracy of said in vitro method, wherein said validating comprises confirming that the correlation between said potentially active materials and said active materials is statistically significant;

(e) optionally, designating said in vitro methods having statistically significant correlations between the results of said in vitro method and said in vivo method as a validated in vitro method;

(f) optionally, screening a plurality of unknown materials using said validated in vitro method to identify potentially active materials.

Validated in vitro methods can be used to more accurately and efficiently screen for and/or identify active materials. This is because such validated methods can more accurately identify potentially active materials that are also active materials (e.g., the correlation between potentially active materials and active materials is statistically significant).

### DETAILED DESCRIPTION OF THE INVENTION

A. Identifying a Biomarker that is Indicative of Mammalian Keratinous Tissue Regulation

Any suitable method of identifying a biomarker that is indicative of mammalian keratinous tissue regulation can be used herein. In one embodiment, such biomarker is identified by comparing the effects of a material known to regulate the condition of interest (e.g., through prior in vivo testing, through prior in vitro testing, knowledge of the material's function in the art, etc.) with a control. As used herein, a biomarker is a biological parameter in keratinous tissue that is modified as a result of a particular keratinous tissue condition, in comparison to keratinous tissue not having the particular keratinous tissue condition of interest. This modified biological parameter, once identified, can be used to screen for materials that induce the subject positive or negative effect being screened for.

As used herein, “regulate” means inducing and/or causing a positive or negative effect on a mammalian keratinous tissue condition. As used herein, “keratinous tissue condition” means any particular keratinous tissue condition of interest, and can include any keratinous tissue property, both those that are desired as well as those that are undesired. For instance, undesired keratinous tissue conditions can include, but are not limited to, fine lines, wrinkles, dry skin, itchy skin, irritated skin, sensitive skin, rough skin, sallow skin, uneven pigmentation, blotchy skin, skin with an undesired (e.g., uneven) tone, skin with an undesired texture, sundamaged skin, aged skin, oily skin, skin with large pores, etc.

The term “keratinous tissue,” as used herein, refers to keratin-containing layers disposed as the outermost protective covering of mammals which includes, but is not limited to, skin, hair, scalp, nails (e.g., toenails, fingernails), cuticles, hooves, etc.

In a particular embodiment, identifying the biomarker comprises comparing the proteomic and/or transcriptomic and/or genomic analysis of keratinous tissue cells having the condition of interest to a control (e.g., keratinous tissue cells not having the condition of interest). This enables the selection, the identification, and the characterization of potential targets, so as to reverse or to provide an indication of at least one biological parameter modified by the existence of a particular keratinous tissue condition of interest. The potential targets correspond to the biological parameters to be reversed or the modification of which is to be indicated, which are identified by the method of the present invention. After definition of the targets, the same methods of detection can be used for the screening of actives, the demonstration of effectiveness of cosmetic compositions containing the actives, etc.

The cells can be obtained from any suitable source, such as from cells that originate from cell-lines or cells that are of mammalian (e.g., human) origin. In particular embodiments, one or both types of cells (e.g., cells having the condition of interest and/or the control cells) are used in a three-dimensional tissue model. The tissue model should be cultivated and/or preserved under conditions which maintain, at least partially, a cell metabolism.

The biological parameter, which is modified as a result of the condition of interest, should be defined by at least one difference between the metabolism of the unaffected (e.g., control) cells and the metabolism of the affected cells. Any suitable in vitro analytical technique can be used. For instance, for the analysis of the proteomic profile, bidimensional electrophoresis, and/or protein arrays and/or cytokine array, and or combined ELISA can be used. For instance, for the analysis of the genomic profile, DNA arrays, and/or polymerase chain reaction multiplex (PCR-multiplex), and/or polymerase chain reaction (PCR), and/or real time polymerase chain reaction (real time PCR) can be used. For the analysis of the transcriptomic profile, techniques that can be used include RNA arrays, cDNA arrays and/or reverse transcription polymerase chain reaction multiplex (RT-PCR-multiplex) and/or reverse transcription polymerase chain reaction (RT-PCR) and/or real time reverse transcription polymerase chain reaction (real time RT-PCR).

B. Screening In Vitro at Least One Unknown Material, Preferably a Plurality of Unknown Materials, Against Said Biomarker to Identify One or More Potentially Active Materials

After a biomarker from (A) above is identified, one or more unknown materials are screened against said biomarker to identify one or more potentially active materials. As used herein, “unknown material” means a material that has not yet been identified by the method herein as a material than can induce the subject keratinous tissue condition (e.g., an unknown material is not a potentially active material and is not an active material as defined herein). As used herein, “material” includes, but is not limited to, compounds, mixtures of compounds, extracts, cosmetic product formulations, compounds or mixtures of various concentrations, etc. As used herein, “potentially active material” means a material identified by in vitro testing in this step (B) as a material than can induce the subject keratinous tissue condition.

In one embodiment, the screening step comprises: (1) placing said unknown material in contact with the cells having the keratinous tissue condition of interest from step (A) above, for a period of time sufficient to enable said unknown material to act (e.g., produce the subject regulation of the condition of interest); (2) placing said unknown material in contact with the control cells as set forth in step (A) above, for a period of time sufficient to enable said unknown material to act (e.g., produce the subject regulation of the condition of interest); (3) performing proteomic analysis and/or transcriptomic analysis and/or genomic analysis, partial or complete, for making the study of the action of said substance on the cell metabolism of said cells having the keratinous tissue condition of interest; (4) comparing the cell metabolism of said cells having the keratinous tissue condition of interest in the presence of the unknown substance, with the metabolism of said cells having the keratinous tissue condition of interest or of the control cells without the presence of said unknown material; and (5) identifying the presence or the absence of activity of said unknown material, wherein said identifying step comprises identifying a positive or negative effect of said substance in order to provide an indication of the modification of the biological parameter identified as being modified in keratinous tissue having the subject keratinous tissue condition. In a specific embodiment, the cells of steps (1) and steps (2) are sown in a cell model or tissue model.

In another embodiment, the screening step comprises: (1) culturing the control cells; (2) culturing cells having the keratinous tissue condition of interest, having a biological parameter modified with respect to the control cells, in the presence of at least one unknown material, for a period of time sufficient to enable said unknown material to act on the cell metabolism of said cells, so as to recover the metabolism of the control cells; (3) performing proteomic analysis and/or transcriptomic analysis and/or genomic analysis, partial or complete, of the cells having the keratinous tissue condition which are cultivated in the presence or not of a known active material; (4) comparing the analysis carried out in (3) with the proteomic analysis and/or transcriptomic analysis and/or genomic analysis, partial or complete, of control cells, which are cultivated without the presence of said unknown material; and (5) following the comparison of the analyses carried out in (3) and (4), eventually identifying at least one potentially active material capable of reversing at least one biological parameter identified as being modified in keratinous tissue having the subject keratinous tissue condition.

In yet another embodiment, the screening step comprises identifying at least one potentially active material capable of providing an indication of the modification of at least one biological parameter modified in keratinous tissue having the subject keratinous tissue condition compared to the control. In this embodiment, the steps of this screening step comprise: (1) placing said unknown material in contact with the cells having the keratinous tissue condition of interest from step (A) above, for a period of time sufficient to enable said unknown material to act (e.g., produce the subject regulation of the condition of interest); (2) performing proteomic analysis and/or transcriptomic analysis and/or genomic analysis, partial or complete, of the cells having the keratinous tissue condition, placed in contact with the unknown material (3) comparing the analysis carried out in (2) with the proteomic analysis and/or transcriptomic analysis and/or genomic analysis, partial or complete, of living cells which are cultivated without the presence of said unknown material; and (4) following the comparison of the analyses carried out in (3), eventually identifying at least one potentially active material capable of providing an indication of the modification of at least one biological parameter modified in keratinous tissue having the subject keratinous tissue condition as compared to the control. In a specific embodiment, the cells of step (1) are sown in a cell model or tissue model.

C. Testing Said One or More Potentially Active Materials In Vivo to Confirm the Activity of Said Potentially Active Materials in Order to Identify One or More Active Materials

The potentially active material(s) identified in step (B) above are then tested in vivo to confirm the activity of said potentially active materials identified in vitro. Those potentially active materials which are shown through in vivo testing to regulate the keratinous material condition that was shown to be regulated in the in vitro testing of step (B) are designated as active materials.

Any suitable in vivo test method can be used, including those known in the art.

D. Optionally, Validating the Accuracy of Said In Vitro Method, Wherein Said Validating Comprises Confirming that the Correlation Between Said Potentially Active Materials and Said Active Materials is Statistically Significant

By utilizing statistical calculations known in the art, the accuracy of the in vitro method can be validated by confirming that the correlation between those materials identified as potentially active (through in vitro testing) and those ultimately identified as active materials (through in vivo testing) is statistically significant. Confirming that the subject in vitro test gives substantially the same results as the in vivo test can reduce the time and effort expended, by reducing and/or eliminating the number of materials that show the desired activity in vitro, but do not deliver the desired activity in vivo. Furthermore, in some cases, using such a validated method can even replace (e.g., substitute for) the use of in vivo testing altogether. When an in vitro test method is shown to correlate in a statistically significant manner to in vitro testing, the in vitro test method can be used, in many instances, for example, to support advertising claims, marketing claims, efficacy claims, etc., as well as to provide safety data (e.g., preliminary).

E. Optionally, Designating Said In Vitro Methods Having Statistically Significant Correlations Between the Results of Said In Vitro Method and Said In Vivo Method as a Validated In Vitro Method

The in vitro methods validated as having statistically significant correlations between the results of said in vitro method and said in vivo method in step (D) above can be designated as “validated” in vitro methods.

F. Optionally, Screening a Plurality of Unknown Materials Using Said Validated In Vitro Method to Identify Potentially Active Materials

Once an in vitro method is validated, it can be used to identify potentially active materials. Such potentially active materials can optionally then be designated as “selected materials.” As used herein, “selected materials” means, and can include one or more, or a combination of one or more: (1) active materials, and/or (2) potentially active materials identified by a validated in vitro method.

### Cosmetic Compositions

In another embodiment, the invention can relate to selected materials. In yet another aspect, the invention relates to cosmetic compositions comprising at least one selected material.

Such cosmetic compositions can be in any suitable form. All forms of topical and oral personal care compositions are contemplated and can include, for instance, creams, gels, lotions, emulsions, colloids, solutions, suspensions, ointments, milks, sprays, capsules, tablets, liquids, sticks, solids, powders, compacts, pencils, spray-on formulations, brush-on formulations, cloths, wipes, and the like. Non-limiting examples of topical cosmetic compositions can include, without limitation, lipstick, mascara, rouge, foundation, blush, eyeliner, lipliner, lip gloss, facial or body powder, sunscreens and blocks, nail polish, mousse, sprays, styling gels, nail conditioner, bath and shower gels, shampoos, conditioners, cream rinses, hair dyes and coloring products, leave-on conditioners, sunscreens and sunblocks, lip balms, skin conditioners, cold creams, moisturizers, hair sprays, soaps, body scrubs, exfoliants, astringents, depilatories and permanent waving solutions, antidandruff formulations, antisweat and antiperspirant compositions, shaving, preshaving and after shaving products, moisturizers, deodorants, cold creams, cleansers, skin gels, and rinses.

The term “topical application,” as used herein, means to apply or spread the compositions of the present invention onto the surface of the keratinous tissue.

The term “safe and effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The term “post-inflammatory hyperpigmentation” as used herein refers to the changes in melanin content as a response to an inflammatory event (e.g., acne, scratch, insect sting or bite, sunburn, etc.), especially in dark skin subjects.

The term “hyperpigmentation” as used herein refers to an area of skin wherein the pigmentation is greater than that of an adjacent area of skin (e.g., a pigment spot, an age spot, and the like).

The terms “desquamation, exfoliation, and/or turnover” as used herein mean the removal of the upper layers of the stratum corneum (comprising the horny layers).

The terms “oily and/or shiny appearance” as used herein mean the glossy look mammalian skin tends to exhibit upon the excretion of oil, sebum, and/or sweat from the respective source gland.

The term “sagging” as used herein means the laxity, slackness, or the like condition of skin that occurs as a result of loss of, damage to, alterations to, and/or abnormalities in dermal elastin.

The term “smoothing” and “softening” as used herein means altering the surface of the keratinous tissue such that its tactile feel is improved.

The term “sallowness” as used herein means the pale color, yellow color or the like condition of skin that occurs as a result of a loss of, damage to, alterations to, and/or abnormalities in skin components such that they become colored (e.g., yellow in color) due to processes such as protein glycation and accumulation of lipofuscin or in the decrease in peripheral blood flow that typically accompanies skin aging.

Cosmetic compositions of the present invention can be useful for topical application and/or for oral administration, for regulating keratinous tissue condition. Regulation of keratinous tissue condition, especially human skin condition, is often desired due to conditions that may be induced or caused by factors internal and/or external to the body. For instance, “regulating skin condition” can include prophylactically regulating and/or therapeutically regulating skin condition, and may involve one or more of the following benefits: moisturizing and/or hydrating skin; thickening (i.e., building the epidermis and/or dermis layers of the skin and/or the subcutaneous layers such as fat and muscle and where applicable the keratinous layers of the nail and hair shaft) to reduce atrophy (e.g., of the skin), increasing the convolution of the dermal-epidermal border, non-melanin skin discoloration such as under eye circles, blotching (e.g., uneven red coloration due to, e.g., rosacea) (hereinafter referred to as “red blotchiness”), sallowness (pale or yellow color), discoloration caused by telangiectasia or spider vessels, discolorations due to melanin (e.g., pigment spots, age spots, uneven pigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, flakes, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin. Regulating skin condition can also involve improving skin appearance and/or feel. Regulating keratinous tissue condition can comprise regulation of any condition set forth and/or defined herein, as well as other conditions not expressly set forth herein.

As used herein, “regulating skin condition” is intended to include regulation of such signs irrespective of the mechanism of origin. In one embodiment, regulating skin condition comprises regulation of those signs commonly associated with the signs of aging.

### Methods of Doing Business

In another aspect, the present invention also relates to methods of doing business. Such methods can include, but are not limited to, communicating safety and/or efficacy data obtained through use of the method herein to support marketing claims and/or advertising claims, and to provide irritation and/or safety information (e.g., data). In addition, such methods can include communicating the mechanism by which active materials or potentially active materials (e.g., those that are identified using a verified method), and/or cosmetic compositions comprising such materials, function to regulate a particular keratinous tissue condition. This information can be communicated, for example, to the public (e.g., via advertising) or to regulatory agencies. As used herein, the public can include, but is not limited to, purchasers and/or potential purchasers of a cosmetic composition.

In one embodiment, the method of doing business of the present invention comprises: (1) offering for sale a cosmetic composition comprising a selected material; (2) optionally, communicating information related to said composition and/or selected material to the public and/or a regulatory agency, wherein said information comprises data obtained from the method for identifying materials that can help regulate mammalian keratinous tissue condition herein.

### EXAMPLES

### Example 1

### Hyaluronic Acid and Collagen Example

Objective:

To evaluate skin anti-aging benefits of n-acetyl glucosamine and niacinamide. An in vitro skin biomarker experiment is conducted using the MatTek Human Skin EpiDermFT Skin Model (EFT 200; MatTek Corp., Ashland, Mass. USA). The MatTek Skin Model is a full thickness culture system that emulates normal human skin properties and function. It contains a three-dimensional, highly differentiated human epidermis with 8-12 cell layers including basal, spinous, granular and stratum corneum layers; the epidermis is grown above a human dermal fibroblast-containing collagen matrix. The MatTek cultures are air-interfaced, so that test substances can be applied topically to the stratum corneum surface. The cultures are supplied in 24-well, medium-supplemented agarose gels.

Methodology:

MatTek EFT-200 full thickness skin equivalent cultures used are examined for visual defects and equilibrated overnight after their arrival from the supplier. After treatment with test materials the cultures' viability is confirmed through MTT viability assay.

Experimental Treatments=Vehicle Control (water)
0.5% n-Acetyl Glucosamine
1.0% n-Acetyl Glucosamine
2.0% n-Acetyl Glucosamine

After treatment for 20-24 hours, replicate (3) cultures are extracted using a mild extraction buffer (T-per). The extract is then analyzed for hyaluronic acid and procollagen-1.

Apparatus:

Kontes Glass Tissue Grinder

Micro centrifuge

Spectraflour Plus

Bioplex

Ice Block −20° C.

Reagents:

EFT-200 full thickness skin equivalent cultures, MatTek

EFT-200 Assay Maintenance Media, MatTek

MTT-200 Kit, MatTek

T-Per, Pierce

Micro BCA Protein Assay, Pierce

Procollagen I ELISA, Takara Bio Inc.

Hyaluronic Acid ELISA, Corgenix

Procedure:

1. Treatment of Cultures: Feed cultures by pipetting 2 mL/well of fresh pre-warmed medium into bottom of six well plates, then transfer cultures to plates. Topically treat cultures with 100 μL/culture of treatment compounds and place in incubator at 37° C., 5% CO₂, 95% RH (relative humidity) for 20-24 hrs.
2. Preparation of Tissue: Rinse off treatment with PBS (phosphate buffered saline)
3. Protein Extraction: Remove skin cultures from snap well and place in 1.5 mL centrifuge tubes on ice block. Incubate cultures with 300 μL of T-per for 30 minutes. Homogenize cultures, centrifuge mixture at 10,000 rpm (revolutions per minute) for 5 minutes, then collect supernatant. Analyze supernatant with the Micro BCA kit to quantitate protein concentration.
4. Hyaluronic Acid ELISA: Dilute protein samples 1:300 in assay buffer. Run protocol following package insert on ELISA kit.
5. Procollagen I ELISA: Dilute protein samples 1:5 in assay buffer (final concentration 4 μL protein per assay well). Run protocol following package insert on ELISA kit.
   Biomarkers of Interest
Hyaluronic Acid (HA) is a highly conserved extracellular glycosaminoglycan. HA is a linear polysaccharide composed of a repeating disaccharide of N-acetyl-D-glucosamine-β and D-glucoronic acid-β residues. HA serves several crucial physiologic functions including barrier effects, cell proliferation and migration, tissue resiliency and elasticity, wound healing and overall hydration.
Procollagen I (the precursor to Collagen I) gives rise to the main constituent of tissue formation in the dermis. “The main types of collagen (Col) found in connective tissue types are types 1, 2, 9, 10 & 11 with Col 1 being the principal collagen of skin and bone and by far the most common.” Source: Expression of collagen and fiber-associated proteins in human septal cartilage during in-vitro dedifferentiation, Goessler, U. R., Bugert, P. et. al., International Journal of Molecular Medicine 14: 1015-1022, 2004.
   Results:

The results indicate that treatment of the cultures with n-Acetyl Glucosamine (NAG) increase expression of hyaluronic acid and procollagen 1 in a dose-responsive manner, to levels significantly increased over Control.

Summary:

The biomarker results indicate that NAG produces increased hydration of the skin cultures via increased hyaluronic acid expression. This also leads to increased procollagen 1 expression. Both of these responses correspond to projected anti-aging facial benefits.

### Example 2

In Vitro Modeling of Skin Pigmentation Effects:

Experimental Objectives:

The objective of this model is to determine the capacity of selected test materials of diverse mechanistic capability, and their combinations, to reduce melanin production. The results are relevant to the clinical reduction of age spots and other unwanted skin pigmentation.

Culture and Treatment of Pigmented Epidermal Cultures

Pigmented skin equivalent cultures (RHTE Type VI, SkinEthic, France) are used to evaluate the ability of materials of interest in the reduction of pigmentation production. Cultures are received at Day 11-13 of culture, and are equilibrated overnight after receipt in the manufacturer's supplied medium, using standard tissue culture techniques. The following day, Day 0, a group of cultures are harvested and stored frozen to quantify the basal level of melanin. Remaining cultures are treated topically, or through the test medium with the materials of interest, or combinations of these materials. Medium and test treatment is replenished daily throughout the experiment.

Viability assessment: On Day 7 of the experiment, the cultures are evaluated for their viability using the MTT assay. The treatment is removed from each culture and each culture is placed into a tissue culture plate containing MTT (Thiazolyl Blue Tetrazolium Bromide, Sigma Chemicals) in Advanced DMEM (Invitrogen). The cultures are then incubated for three hours to allow for uptake and metabolism of the MTT. Following the MTT uptake, each culture is blotted with paper toweling and transferred to a tissue culture plate containing isopropanol, where the metabolized MTT dye is extracted from the culture. The extractant from this step is read using a microplate reader equipped with a λ=570 filter (VMax, Molecular Devices Corp., or equivalent). The resulting readings of the extractant are compared to the readings from an appropriate vehicle control group to calculate the % Control Viability.

Melanin Quantification:

The culture remaining following the MTT is removed from its tissue culture insert and analyzed for melanin. Each culture is incubated in a tissue solubilizer, Solvable (Packard Chemicals) at 70° overnight. Residual undissolved tissue is removed by centrifugation, and the resulting supernate is read on a microplate reader equipped with a λ=405 filter (VMax, Molecular Devices Corp., or equivalent). Readings from each sample are compared to readings generated from a standard curve of synthetic melanin (Sigma Chemicals) for quantification. The reading from the baseline cultures is subtracted to obtain the change in melanin for each culture. This value is then corrected for viability changes that may have affected melanin production by dividing by the % Control Viability.

### Example 3

In Vitro Modeling of Skin Sensory Effects:

Experimental Objectives & Design:

The objective of this model is to determine the potential of cosmetic preparations, such as hair colorants, to produce adverse sensory responses in the skin. This determination is made by comparing the cytokine release and viability of cultures treated with materials of known clinical response (high and low controls) with those of unknown clinical response.

Interference Testings:

To determine if the test material interferes with MTT reduction used for viability measures or the mesh used to spread the test material, interference tests must be conducted. The test material is prepared as described below, and applied (40 uL) to a piece of nylon mesh disk (Spectrum Labs) which is cut to the size of the culture using an 8 mm biopsy punch (Miltex Instruments). The disk is incubated at 37° C. for one hour (or longer, depending on desired incubation time). Following incubation, the disk is rinsed clean with PBS and placed on a glass slide. The disk is compared microscopically to an untreated disk to determine if the treatment product has degraded the mesh. If the disk is visibly degraded following this test, the test material must be spread manually on the culture surface with the pipet tip.

For the MTT interference test, 40 μL of the test material is pipetted directly into a well of a 24-well tissue culture plate containing 300 μL of MTT (Sigma Chemicals). A control well containing MTT but no test material is included in the plate. The plate is incubated at 37° C. for one hour. Following incubation, the test well is compared visually to the control well for color changes. If bleaching of the well or development of color occurs in the test well that is not observed in the control well, a freeze-killed control culture must be included in the assay for both test and control groups.

Culture of Epidermal Cultures

Skin equivalent cultures (EpiDerm 200, MatTek Corp., Boston, Mass.) are used to evaluate cytokine release upon exposure to hair dyes, or other materials of interest. Cultures are received at Day 11 of culture, and are equilibrated overnight after receipt in the manufacturer's supplied medium (EPI100-HCF-PRF), using standard tissue culture techniques. The following day, the medium is changed prior to treatment of the cultures.

Treatment Preparation:

Commercially packaged hair colorants, and hair colorants for clinical and consumer testing, are packaged in two separate containers, containing the colorant and developer. The ready-to-use hair colorant is prepared by pouring or squeezing the contents of the colorant container into the bottle which contains the developer. The developer bottle is capped and shaken vigorously by hand for approximately 20 seconds. Laboratory prepared colorants may be mixed by the above method or by combining colorant with developer in a glass beaker, and stirring for 20-30 seconds with a glass stirring rod. The mixture is evaluated visually to insure that the mixture is homogeneous, with no visible swirls of darker or lighter material. All preparations are applied to the cultures within 10 minutes of preparation, and are not reused.

Treatment Application & Culture Harvest:

Treatments are applied topically (40 μL) to each culture using a positive displacement pipettor (Rainin). The treatment is distributed across the surface of the culture by placing an 8 mm nylon mesh disk on the surface of the treatment, and gently pressing with sterile blunt forceps. The time of each application is recorded. Following treatment application, the cultures are incubated at 37° C., 5% CO₂, 90-95% humidity inside a tissue culture incubator.

Freeze-Killed Cultures:

In many cases a freeze-killed culture (EPI-200-FRZN, MatTek Corp) is used to correct for interference of items that have been determined to reduce MTT (see above). In this case, the freeze-killed culture should be removed from −20° C. storage and placed in a well of medium on a plate with the other cultures in its treatment group prior to the beginning of treatment. The freeze-killed culture should be treated exactly the same as all the other cultures in the group. If freeze-killed cultures are used for any group within the study, a freeze-killed culture must be used for the control as well.

Viability Assessment (MTT Assay):

Test materials and the nylon mesh disk are thoroughly rinsed from each culture using Dulbecco's PBS (Invitrogen) at its designated rinse time and discarded. The culture is placed into a tissue culture plate containing 1 mg/mL MTT (Thiazolyl Blue Tetrazolium Bromide, Sigma Chemicals) in Advanced DMEM (Invitrogen). The cultures are then incubated for three hours to allow for uptake and metabolism of the MTT. Following the MTT uptake, each culture is blotted with paper toweling and transferred to a tissue culture plate containing isopropanol, where the metabolized MTT dye is extracted from the culture. The extractant from this step is read using a microplate reader equipped with a λ=570 filter (VMax, Molecular Devices Corp., or equivalent). The resulting readings of the extractant are divided by the readings from a vehicle control group to calculate the % Control Viability incubator for one hour. If killed controls are used for the experiment, the absorbance resulting from the extractant of the killed control for each group is subtracted prior to the calculation.

Harvest of Medium and ELISA Procedures:

Once exposure to test materials is complete and cultures enter the MTT assay, the media is harvested into 96-well tube racks (National Scientific) and stored at 4° C. for subsequent ELISAs. Samples of IL1a, IL8 or IL1RA may be stored at 4° C. for up to one week with no compromise in stability or at −20° C. to −80° C. for longer storage. Note that it is not necessary to harvest the media from freeze-killed cultures.

The IL8 and IL1α ELISAs (R&D Systems) are performed as described in the manufacturer's protocol using the undiluted culture medium from each sample. Samples are typically assayed in singlet (one culture supernate=one ELISA well). The standard curve is typically performed in duplicate. The IL1RA ELISA (R&D Systems) is performed as described in the manufacturer's protocol, with the exception that samples are diluted 1:10 dilution in the calibrator diluent (provided with the ELISA kit) prior to addition to the ELISA to insure that the samples fall within the range of the standard curve. Cytokine concentrations are obtained by performing a linear regression analysis, or four parameter curve fit, using the standard curve samples and calculating the concentration of each unknown using the parameters generated by the standard curve. Each cytokine concentration is then divided by the % Control Viability to correct for culture viability changes during the incubation. The result is referred to as the normalized cytokine concentration, which directly correlates to the material's clinical score for Transient Sensory Response (TSR, or sting). Note that higher TSR scores are indicative of more stinging, thus less consumer acceptability. Materials with TSR scores equal to or lower than the high TSR control are acceptable for further evaluation. Results of IL1α, IL1RA, the IL1RA ratio, as well as IL8 have been shown to have predictive value in this testing, although IL8 is the most consistently predictive.

### Example 4

In Vitro Measurement of Transepidermal Water Loss (TEWL)

Experimental Objectives:

The objective of this model is to determine the capacity of selected test materials to increase or maintain the barrier of skin.

Culture and Treatment of Human Skin Equivalents:

Human skin equivalents from any supplier can be measured using this method. The skin equivalents are cultured according to the manufacturer's protocol, using the manufacturer's supplied medium and standard tissue culture techniques. Test materials are administered either to the apical surface of the culture or through the tissue culture medium.

TEWL Measurement:

TEWL measurement is performed prior to any treatment of the cultures and at designated days during the treatment period, using a VapoMeter SW (Delfin Technologies Lt., Finland). The skin adapter of this instrument is removed to enable a tight fit of the measurement chamber over 8-10 mm tissue culture inserts. Other cultures which are not provided in this insert may be measured by directly placing the VapoMeter (with skin adapter) on the apical surface of the culture, or by creating an adaptor by which the VapoMeter chamber may be sealed against the tissue culture insert. Prior to measurement, cultures are removed from the tissue culture incubator and placed with the lids of their plates off inside a biological safety cabinet. Moisture on the apical surface of the culture or its insert is removed by blotting gently with a sterile swab. Cultures are equilibrated to temperature and humidity conditions inside the biological safety cabinet for at least 20 minutes prior to measurement. Measurements are collected from cultures using a randomized order to minimize effects of environmental changes and instrumental drift. Each culture insert is removed individually from its well and placed onto a square of medium-soaked sterile gauze in a Petri dish. TEWL measurement is recorded by placing the Vapometer over the tissue culture insert, so that the sides of the insert are inside the walls of the Vapometer chamber. Measurements are made in standard mode of the instrument. At least two consecutive measurements are made for each culture.

Except as otherwise noted, all amounts including quantities, percentages, portions, and proportions, are understood to be modified by the word “about,” and amounts are not intended to indicate significant digits.

All percentages and ratios used herein are by weight of the total composition and all measurements made are at 25° C., unless otherwise designated.

Except as otherwise noted, the articles “a,” “an,” and “the” mean “one or more.”

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited herein are incorporated herein by reference in their entirety; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for identifying active materials that can regulate the condition of mammalian keratinous tissue, wherein said method comprises the steps of:
(a) identifying a biomarker that is indicative of mammalian keratinous tissue regulation;
(b) screening in vitro at least one unknown material, preferably a plurality of unknown materials, against said biomarker to identify one or more potentially active materials;
(c) testing said one or more potentially active materials in vivo to confirm the activity of said potentially active materials in order to identify one or more active materials;
(d) optionally, validating the accuracy of said in vitro method, wherein said validating comprises confirming that the correlation between said potentially active materials and said active materials is statistically significant;
(e) optionally, designating said in vitro methods having statistically significant correlations between the results of said in vitro method and said in vivo method as a validated in vitro method;
(f) optionally, screening a plurality of unknown materials using said validated in vitro method to identify potentially active materials.

2. An active material identified by the method of claim 1.

3. A potentially active material identified in step (f) of the method of claim 1.

4. A cosmetic composition comprising the active material of claim 2.

5. A cosmetic composition comprising the potentially active material of claim 3.

6. A method of doing business, wherein said method comprises communicating data generated from the method of claim 1 to the public or to a regulatory agency.
